(19) United States
(12) Patent Application Publication
(10) Pub. No.: US 2023/0056496 A1
(43) Pub. Date: Feb. 23, 2023

(54) DETERMINATION OF FETAL GENOTYPE USING MATERNAL BIOLOGICAL SAMPLE

(71) Applicant: Luna Genetics, Inc., Houston, TX (US)

(72) Inventor: Arthur BEAUDET, Houston, TX (US)

(21) Appl. No.: 17/796,603

(22) PCT Filed: Jan. 28, 2021
(86) PCT No.: PCT/US2021/015448

§ 371 Date: Jul. 29, 2022

Related U.S. Application Data
(60) Provisional application No. 62/968,987, filed on Jan. 31, 2020.

(51) Int. Cl. G01N 33/552; C12N 5/073; G01N 33/569; C12Q 1/6883

(52) U.S. Cl. CPC G01N 33/552; C12N 5/0603; C12Q 1/6883; G01N 33/56966; C12Q 2600/156; G01N 2800/385

(57) ABSTRACT

Populations of cells enriched in fetal cells from a biological sample obtained from a pregnant subject are prepared using microbubbles, resulting in a sufficient number of fetal cells having a quality suitable for sequencing and providing non-invasive prenatal diagnosis of genetic disorders.

### CROSS-REFERENCES TO RELATED APPLICATIONS

This application claims the benefit of priority to U.S. Provisional Pat. Application No. 62/968,987, filed on Jan. 31, 2020, the entire contents of which are herein incorporated by reference in its entirety.

### FIELD

This disclosure relates to methods and systems for isolating fetal cells from maternal biological samples, obtaining fetal DNA, and providing a fetal genotype through non-invasive means.

### BACKGROUND

The current best methods for testing for genetic abnormalities in the fetus are amniocentesis and chorionic villus sampling (CVS). In amniocentesis, a sample of amniotic fluid is drawn by needle, and free-floating fetal cells in the fluid are examined. Although this provides a definitive source of fetal DNA, the procedure carries a risk of miscarriage, variously reported at 0.25-0.50% (US Centers for Disease Control); 0.30% (L.J. Salomon et al., Ultrasound Obstet Gynecol (2019) 54(4):442-51; 0.35% (J. Beta et al., Minerva Ginecol (2018) 70(2):215-19), and 0.81% (R. Akolekar et al., Ultrasound Obstet Gynecol (2015) 45:16-26). Amniocentesis is usually performed in the second trimester, between weeks 15 and 20; “early” amniocentesis (performed before gestation week 15) carries a higher risk of complications.

In CVS, a tissue sample is taken from the placenta and examined. Although this tissue derives from the embryo, it sometimes differs from the fetus due to confined placental mosaicism (CPM), in which some of the placental cells are abnormal (about 1-2% of pregnancies). CVS carries a higher risk of complications (estimated at 1-2%), but can be performed earlier than amniocentesis, usually at 10-12 weeks gestation (late in the first trimester).

Cell-free DNA (cfDNA) non-invasive prenatal testing (NIPT) relies on the detection and characterization of extracellular fetal DNA circulating in maternal blood. Cell-free fetal DNA (cffDNA) constitutes about 5-20% of the total cfDNA in a pregnant human, and derives from trophoblasts (placental cells). The cffDNA itself is found in the form of fragments, about 200 base pairs (bp) in length. These characteristics limit the accuracy and utility of cffDNA NIPT, and maternal malignancies, maternal chromosomal mosaicism, CPM, maternal copy number variants (CNVs), maternal body mass index (BMI), and maternal organ transplants can substantially affect the data. This can compromise the test result, leading to false positive aneuploidy or CNV results, and sometimes false negative results. Hence, cell-free NIPT is considered a screening test that requires diagnostic confirmation (i.e., amniocentesis or CVS). cffDNA NIPT fails to detect many genetic defects, such as deletions and unbalanced translocations that would normally be detected by amniocentesis or CVS. The growing use of cffDNA NIPT has led to a reduction in the number of analyses by amniocentesis or CVS, which is expected to result in an increase in the number of children born with cytogenetic abnormalities (L. Hui et al., Annu Rev Med (2017) 68:459-72; A.M. Breman et al., Prenat Diagnos (2016) 36:1009-19).

In contrast to cffDNA non-invasive testing, cell-based NIPT has the selectivity and specificity to become a diagnostic test. The primary challenge for cell-based NIPT is that the target cells are exceedingly rare at 1-2 cells/mL maternal blood (K. Krabchi et al., Clin Genet (2001) 60:145-50), and there is considerable inter-individual variability in the number of recoverable cells. Additionally, not all cells are suitable for use, as cells that are undergoing apoptosis or that are in S phase contain different amounts of DNA, which interferes with attempts to determine smaller copy number variations such as subchromosomal deletions or duplications.

Thus, there exists a medical need for non-invasive prenatal testing that has low or no risk of complications, with accuracy comparable or equal to amniocentesis, which can be reliably performed at a gestational time earlier than amniocentesis.

### SUMMARY

This disclosure presents an improved method of isolating fetal cells from a maternal sample during pregnancy, which reliably and consistently provides a sufficient number of fetal cells in a condition that permits advanced analysis and diagnosis. The fetal cells thus obtained enable the performance of cell-based NIPT, which can determine cytogenetic conditions such as aneuploidy, partial aneuploidy (copy number variations in a portion of a chromosome, as small as about 1 Mb or less), insertions and deletions (indels), translocations, uniparental disomy, single nucleotide polymorphisms (SNPs), and the presence of alleles associated with a pathological condition. This method can be performed before week 15 gestation, and as early as 7 weeks gestation, and carries no risk of complications to the fetus.

One aspect of the disclosure is an improved method for enriching a cell population containing circulating fetal cells so that an adequate number of fetal cells in good condition can be identified and tested.

One aspect of the disclosure is a method for preparing a cell population enriched in fetal cells, by (a) providing a biological sample from a pregnant subject; (b) contacting the sample with (i) at least one binding agent, wherein the binding agent comprises a targeting moiety specific for a fetal cell antigen; and (ii) a buoyant microbubble, wherein the binding agent binds to the microbubble surface, to form fetal cell-binding agent-microbubble complexes, wherein the complexes have an average ratio of fetal cell to microbubble of about 1:5 to about 5:1, and have an average density of from about 40% to about 80% of the average density of the sample; (c) separating the fetal cell-binding agent-microbubble complexes from other cells in the sample; and (d) collecting the fetal cell-binding agent-microbubble complexes from the sample, to provide a cell population enriched in fetal cells.

In an embodiment, step (d) further comprises releasing fetal cells from the complexes. In some embodiments, the fetal cell is a trophoblast. In some embodiments, the fetal cell is a nucleated fetal red blood cell.

In some embodiments, the binding agent further comprises a first linking moiety, and the microbubble further comprises a second linking moiety, where the first linking moiety and the second linking moiety specifically bind to each other. In some embodiments, the first linking moiety and the second linking moiety have a dissociation constant (K_{D}) for each other of from about 10⁻⁶ to about 10⁻¹⁶. In some embodiments, the K_{D} is from about 10⁻⁸ to about 10⁻¹⁵. In some embodiments, the first linking moiety and the second linking moiety are each selected from the group consisting of antibodies, antibody derivatives, antigens, biotin, avidin, and streptavidin. In some embodiments, the first linking moiety and the second linking moiety are each selected from the group consisting of biotin, avidin, and streptavidin. In some embodiments, the second linking moiety comprises an antibody or antibody derivative, and the first linking moiety is an epitope on the targeting moiety.

In some embodiments, the microbubble has a diameter between about 10 µm and about 20 µm. In some embodiments, the microbubble has a diameter between about 13 µm and about 19 µm. In some embodiments, the microbubble has a diameter between about 16 µm and about 18 µm. In some embodiments, the microbubble has a density of about 0.4 g/cm³ and about 0.8 g/cm³. In some embodiments, the microbubble has a density of about 0.6 g/cm³. In some embodiments, the microbubble is hollow. In some embodiments, the microbubble comprises glass. In some embodiments, the fetal cell-binding agent-microbubble complex rises in the sample at a rate of about 1 mm/min to about 15 mm/min at 1 × g. In some embodiments, the fetal cell-binding agent-microbubble complex rises in the sample at a rate of about 5 mm/min to about 10 mm/min at 1 × g.

In some embodiments, step (c) comprises centrifuging the sample. In some embodiments, the sample is centrifuged at about 200 × g to about 800 × g. In some embodiments, the sample is centrifuged at about 400 × g to about 500 × g. In some embodiments, the sample is centrifuged for about 2 minutes to about 10 minutes. In some embodiments, the sample is centrifuged for about 5 minutes.

In some embodiments, the biological sample comprises a blood sample. In some embodiments, the biological sample comprises a cervical secretion. In some embodiments, the biological sample comprises amniotic fluid.

In some embodiments, step (c) comprises removing erythrocytes from the sample by lysis. In some embodiments, the erythrocytes are lysed using ammonium chloride or a surfactant. In an embodiment, the erythrocytes are lysed using Triton X-100®.

In some embodiments, the targeting moiety comprises an antibody. In some embodiments, the antibody is specific for an antigen selected from the group consisting of HLA-G, EpCAM, and TROP-2. In some embodiments, step (c) comprises contacting the sample with at least two binding agents having different targets. In some embodiments, step (c) comprises contacting the sample with a plurality of binding agents having different targets. In some embodiments, binding agents specific for each of HLA-G, EpCAM, and TROP-2 are used.

In some embodiments, the microbubble is first contacted with a binding agent to form a binding agent-microbubble complex, and the fetal cell is then contacted with the binding agent-microbubble complex to form a fetal cell-binding agent-microbubble complex. In some embodiments, the fetal cell is first contacted with a binding agent to form a fetal cell-binding agent complex, and then the fetal cell-binding agent complex is contacted with a microbubble to form a fetal cell-binding agent-microbubble complex. In some embodiments, the sample is contacted with a label. In some embodiments, the label is selected from the group consisting of: 4',6-diamidino-2-phenylindole (DAPI) or a labeled binding agent specific for an antigen selected from the group consisting of HLA-E, HLA-G, MCAM, ATG9B, EpCAM, TROP-2, CD144, CD47, transferrin receptor (CD71), thrombospondin receptor (CD36), glycophorin A, CD147, and CD45. In an embodiment, the label is DAPI or a labeled binding agent specific for an antigen selected from the group consisting of cytokeratin and CD45. In some embodiments, wherein the sample is contacted with a label after step (c).

In some embodiments, step (d) comprises aspirating the fetal cell-binding agent-microbubble complex from the sample surface. In some embodiments, step (d) further comprises diluting the fetal cell-binding agent-microbubble complex to provide aliquots having 0 or 1 cells. In some embodiments, the aliquots are contained in separate wells. In some embodiments, the aliquots are contained in separate droplets. In some embodiments, step (d) further comprises isolating the fetal cell using a single-cell picking device. In some embodiments, step (d) further comprises isolating the fetal cell using a microfluidic device.

In some embodiments, the cell population enriched in fetal cells has a ratio of fetal cell to other cells of at least about 1:100, at least about 1:20, at least about 1:10, at least about 1:5, about 1:1, or greater than about 1:1. In some embodiments, the biological sample is obtained from the subject at a gestational age of less than about 17, 16, 15, 14, 13, 12, 11, 10, 9, 8, 7, 6, 5, or 4 weeks. In some embodiments, the gestational age is between about 6 weeks and about 15 weeks. In some embodiments, the sample is fixed with formaldehyde or glutaraldehyde.

In some embodiments, the number of fetal cells reliably obtained is at least about 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 80, or 100 fetal cells per sample. In some embodiments, at least about 5, 10, 20, 25, 30, 35, 40, 45, or 50 fetal cells are obtained from at least about 70, 75, 80, 85, 90, 95, 96, 97, 98, or 99% of samples from the subjects tested.

Another aspect of the disclosure is a method for obtaining nucleic acids from fetal cells present in a biological sample obtained during pregnancy, by (a) providing a cell population enriched in fetal cells by any of the methods described herein, and lysing the fetal cells to obtain the nucleic acids. In some embodiments, the fetal cells are lysed as a pool of about 5 fetal cells or fewer. In some embodiments, the fetal cells are lysed as a pool of less than about 4, 3, or 2 fetal cells. In some embodiments, the fetal cells are lysed individually.

In some embodiments, the cell population enriched in fetal cells comprises fetal cells at a ratio of fetal cells to maternal cells of about 1:5 to about 5:1. In some embodiments, the ratio is about 1:1. In some embodiments, both fetal and maternal cells are lysed individually.

Another aspect of the disclosure is a method for genotyping a fetus, by (a) providing fetal cell nucleic acids by the methods described herein, (b) amplifying the nucleic acids; and detecting an indication of a genetic difference. In some embodiments, the indication is a copy number variation of a gene or a chromosomal region. In some embodiments, the chromosomal region is less than about 2 Mb in length. In some embodiments, the chromosomal region is less than about 1, about 0.9, about 0.8, about 0.7, about 0.6, about 0.5, about 0.4, about 0.3, about 0.2, or about 0.1 Mb in length. In some embodiments, the indication is a copy number variation of substantially an entire chromosome. In some embodiments, the indication is a translocation. In some embodiments, the indication is a nucleic acid sequence associated with a pathological condition. In some embodiments, the nucleic acid sequence associated with a pathological condition is an allele associated with a pathological condition. In some embodiments, the indication is a polymorphism. In some embodiments, the polymorphism is an indel. In some embodiments, the indication is a single nucleotide polymorphism (SNP). In some embodiments, the genetic difference is mosaicism. In some embodiments, the mosaicism is confined placental mosaicism. In some embodiments, the genetic difference is uniparental disomy. In some embodiments, the genetic difference is twins.

In some embodiments, the pathological condition is 1p36 deletion syndrome, 18 p deletion syndrome, 21-hydroxylase deficiency, Alpha 1-antitrypsin deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Aicardi-Goutieres syndrome, Albinism, Alexander disease, alkaptonuria, Alport syndrome, Alternating hemiplegia of childhood, Amyotrophic lateral sclerosis - Frontotemporal dementia, Alström syndrome, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Björnstad syndrome, Bloom syndrome, Birt-Hogg-Dube syndrome, Brody myopathy, Brunner syndrome, CADASIL syndrome, CARASIL syndrome, Chronic granulomatous disorder, Campomelic dysplasia, Canavan disease, Carpenter Syndrome, Cerebral dysgenesis-neuropathy-ichthyosis-keratoderma syndrome (SEDNIK), Cystic fibrosis, Charcot-Marie-Tooth disease, CHARGE syndrome, Chediak-Higashi syndrome, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn’s disease, Crouzon syndrome, Crouzonodermoskeletal syndrome (Crouzon syndrome with acanthosis nigricans), Darier’s disease, Dent’s disease (Genetic hypercalciuria), Denys-Drash syndrome, De Grouchy syndrome, Down Syndrome, Di George’s syndrome, Distal hereditary motor neuropathies, multiple types, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich’s ataxia, G6PD deficiency, Galactosemia, Gaucher disease, Gerstmann-Sträussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington’s disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, familial, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer’s syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimeta-physeal dysplasia, Strudwick type), Tay-Sachs disease, Tetrahydrobiopterin deficiency, Thanatophoric dysplasia, Treacher Collins syndrome, Trisomy 8, Trisomy 9, Trisomy, 22, Tuberous sclerosis complex (TSC), Turner syndrome, Usher syndrome, Variegate porphyria, von Hippel-Lindau disease, Waardenburg syndrome, Weissenbacher-Zweymüller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (triple X syndrome), XXXX syndrome (48, XXXX), XXXXX syndrome (49, XXXXX), XYY syndrome (47,XYY), or Zellweger syndrome.

In some embodiments, the pathological condition is selected from the group consisting of: Angelman syndrome, Canavan disease, Charcot-Marie-Tooth disease, Cri du chat syndrome, Cystic fibrosis, DiGeorge syndrome, Down syndrome, Duchenne muscular dystrophy, Familial hypercholesterolemia, Haemochromatosis, Hemophilia, Klinefelter syndrome, Neurofibromatosis, Phenylketonuria, Polycystic kidney disease (PKD1 or PKD2, Prader-Willi syndrome, Sickle cell disease, Spinal muscular atrophy, Tay-Sachs disease, and Turner syndrome.

In some embodiments, the nucleic acids from fetal cells are amplified as a pool of about 5 fetal cells or less. In some embodiments, the pool of fetal cells has about 4, 3, 2, or 1 fetal cell. In some embodiments, the pool of fetal cells has about 1 fetal cell. In some embodiments, step (b) comprises whole genome amplification. In some embodiments, step (c) comprises quantitative polymerase chain reaction amplification (qPCR), array comparative genomic hybridization (array CGH), or next generation sequencing (NGS). In some embodiments, NGS is single cell NGS. In some embodiments, NGS is used at a depth of at least about 20 X. In some embodiments, NGS is used at a depth of at least about 22 X, 23 X, 24 X, 25 X, 26 X, 27 X, 28 X, 29 X, or 30 X.

Another aspect of the disclosure is a system for obtaining nucleic acids from fetal cells present in a maternal biological sample during pregnancy, comprising (a) at least one binding agent, wherein the binding agent comprises a targeting moiety specific for a fetal cell antigen; and (b) a buoyant microbubble, wherein the binding agent binds to the microbubble surface; wherein the binding agent and microbubble form fetal cell-binding agent-microbubble complexes, wherein the complexes have an average ratio of fetal cell to microbubble of about 1:5 to about 5:1, and wherein the microbubble has a density of about 0.4 g/cm³ and about 0.8 g/cm³.

In some embodiments, the system further comprises a label. In some embodiments, the label is specific for a fetal cell. In some embodiments, the microbubble has a diameter between about 10 µm and about 20 µm. In some embodiments, the microbubble has a diameter between about 13 µm and about 19 µm. In some embodiments, the microbubble has a diameter between about 16 µm and about 18 µm. In some embodiments, the microbubble has a density of about 0.6 g/cm³.

Another aspect of the disclosure is a fetal cell composition comprising a fetal cell-binding agent-microbubble complex as described above, and maternal cells, wherein the ratio of fetal cells to maternal cells is from about 1:1,000 to about 1,000:1. In some embodiments, the ratio is from about 1:100 to about 5:1. In some embodiments, the ratio is from about 1:10 to about 1:1. In some embodiments, the fetal cell is a circulating trophoblast or a fetal nucleated red blood cell. In some embodiments, the ratio of fetal cell to microbubble is about 5:1 to about 1:5. In some embodiments, the ratio of fetal cell to microbubble is about 1:1.

Another aspect of the disclosure is a fetal cell-enriched cell population, comprising fetal cells as described herein, and maternal cells, wherein the ratio of fetal cells to maternal cells is from about 1:1,000 to about 1,000:1. In some embodiments, the ratio is from about 1:100 to about 5:1. In some embodiments, the ratio is from about 1:10 to about 10:1. In some embodiments, the fetal cell is a circulating trophoblast or a fetal nucleated red blood cell. In some embodiments, the fetal cells are stained. In some embodiments, the composition is obtained by any of the methods described herein.

The foregoing summary is illustrative only and is not intended to be in any way limiting. In addition to the illustrative embodiments and features described herein, further aspects, embodiments, objects and features of the disclosure will become fully apparent from the detailed description and the claims.

### DETAILED DESCRIPTION OF THE DISCLOSURE

The challenge of isolating fetal cells by non-invasive means to obtain a number sufficient for deep sequencing is overcome by the methods of the disclosure. In these methods, maternal samples containing fetal cells are obtained from maternal blood or cervical samples, and are separated from the rest of the sample by specifically binding the fetal cells to buoyant microbubbles to form complexes that physically separate the fetal cells from maternal cells with high efficiency.

In one method of the disclosure, a cell population enriched in fetal cells is obtained by obtaining a biological sample from a pregnant subject; contacting the sample with at least one binding agent having a targeting moiety, and a buoyant microbubble which can bind the binding agent to its surface; to form fetal cell-binding agent-microbubble complexes, wherein the complexes have an average ratio of fetal cell to microbubble of about 1:5 to about 5:1, and have an average density of from about 40% to about 80% of the average density of the sample; allowing the fetal cell-binding agent-microbubble complexes to separate from other cells in the sample; and collecting the fetal cell-binding agent-microbubble complexes from the sample, to provide a cell population enriched in fetal cells. Fetal nucleic acids are obtained from isolated, individual fetal cells, and are analyzed by single-cell sequencing to enable preparation of a genotype and diagnosis of genetic disorders. The methods are described in more detail below.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all subcombinations of the various embodiments and elements thereof are also specifically embraced by the present disclosure, and are disclosed herein just as if each and every such sub-combination was individually and explicitly disclosed herein.

As used herein, the term “fetal cell” includes cells of direct fetal origin, such as fetal nucleated red blood cells, and other cells derived or descended from the zygote, such as cells of placental or other origin, for example trophoblasts.

### Methods for Obtaining a Cell Population Enriched in Fetal Cells

As set forth herein, amniocentesis and CVS provide valuable genetic information regarding a fetus, but also carry a non-zero risk of complications, including loss of the fetus. Non-invasive prenatal testing based on circulating free fetal DNA (cffDNA) from maternal blood has essentially zero risk, but also has an accuracy insufficiently high to be useful for diagnosis. Circulating fetal cells have been identified in maternal blood, but they are difficult to purify and enrich in a reliable and sufficient quantity. Methods of the disclosure overcome these difficulties by employing a system having a binding agent that specifically binds a fetal cell surface antigen, where the binding agent is linked to a buoyant microbubble, and the microbubble has sufficient buoyancy to enable efficient separation of the fetal cells from the bulk of the maternal cells in a biological sample.

### Binding Agents

Binding agents of the disclosure comprise a targeting moiety that specifically binds to an antigen found on a circulating fetal cell. The target antigen does not need to be unique to fetal cells, but can be any antigen that distinguishes between the target fetal cells and maternal cells present in the biological sample. The target antigen can also be any antigen that distinguishes between circulating fetal cells and maternal cells found in the biological sample after a pre-treatment, for example without limitation, the sample after pre-treatment to remove or lyse maternal red blood cells. The fetal cells most likely to be found in the maternal circulation are trophoblasts and fetal red blood cells, for example fetal nucleated red blood cells (fnRBC). Trophoblasts (placental cells) are believed to enter the bloodstream during the time in which the placenta is forming and invading the uterine wall, after implantation of the fetus. Suitable target antigens for binding trophoblasts include, without limitation, HLA-E, HLA-G, MCAM (CD 146), ATG9B, EpCAM, TROP-2, CD31, CD 141, CD144, MMP9, ITGA1, CSHI, CD105, LVRN, EGFR, ErbB2, ErbB3, ErbB4, annexin A4, and the like. Suitable target antigens for fnRBCs include, without limitation, CD47, transferrin receptor (CD71), thrombospondin receptor (CD36), glycophorin A (CD235a), CD 147, and the like.

Targeting moieties can be any molecule that binds with sufficient specificity and affinity, such as an antibody or an antibody derivative. Antibodies and derivatives useful herein include, without limitation, an antibody with an Fc part or without an Fc part, including a multispecific antibody, a bispecific antibody, a single chain variable fragment (scFv), a tandem scFv, an antibody mimetic such as DARPin, a naked monospecific antibody, a maxibody, a minibody, a nanobody, an intrabody, a diabody, a triabody, a tetrabody, an aptamer, v-NAR, or a camelid antibody. Antigen binding fragments can be grafted into scaffolds based on polypeptides such as fibronectin type III (Fn3) (see, e.g., US 6,703,199). An embodiment is the binding agent wherein the targeting moiety comprises an antibody, an scFv, an Fab, an (Fab)₂, an F(ab)'₂, or a nanobody. Suitable affinities between the target moiety and the target can be, for example, a K_{D} of about 10⁻⁶, 10⁻ ⁷, 10⁻⁸, 10⁻⁹, 10⁻¹⁰, 10⁻¹¹, or 10⁻¹² to its target. An embodiment is the binding agent having a K_{D} of about 10⁻⁸, 10⁻⁹, 10⁻¹⁰, 10⁻¹¹, or 10⁻¹² to its target. An embodiment is the binding agent having a K_{D} of about 10⁻⁸, 10⁻⁹, or 10⁻¹⁰ to its selected target. An embodiment is the binding agent wherein the targeting moiety specifically binds HLA-E, HLA-G, MCAM, ATG9B, EpCAM, TROP-2, CD144, CD47, CD71, CD36, glycophorin A, or CD147. An embodiment is the binding agent wherein the target antigen is HLA-G, MCAM, TROP-2, or CD144. An embodiment is the binding agent wherein the target antigen is CD47, CD71, CD36, glycophorin A, or CD147.

Multiple different binding agents can be used in the methods of the disclosure. For example, binding agents targeting 2, 3, 4, 5, 6, 7, 8, 9, or 10 different target antigens can be employed. In some embodiments, between 1 and 10 different binding agents are used. In some embodiments, between 2 and 8 different binding agents are used. In some embodiments, between 3 and 5 different binding agents are used. Fetal cells of any type can be targeted simultaneously, for example, binding agents specific for trophoblasts and binding agents specific for fnRBCs may be used in the same sample.

Binding agents can be bound to microbubbles directly or indirectly. For example, the binding agent can be coupled chemically directly on a suitable functional group present on the surface of the microbubble. One can employ methods described in the art to attach covalent linking groups to a targeting moiety and a microbubble (see, e.g., K. Tsuchikama et al., Protein Cell (2018) 9(1):33-46; Jablonski et al., US 2011/0236884). Alternatively, the binding agent can comprise a first linking agent which binds specifically to a second linking agent that is bound to the microbubble. The first and second linking moieties can form a covalent or non-covalent bond. The first linking moiety can be, for example without limitation, an epitope that is recognized by the second linking moiety, an additional targeting moiety, or a moiety such as biotin, avidin, or streptavidin. The second linking moiety can also be, for example without limitation, an epitope that is recognized by the first linking moiety, an additional targeting moiety, or a moiety such as biotin, avidin, or streptavidin or their equivalents, where the first and second linking moieties are selected for binding to each other. In an embodiment, the binding agent comprises a first linking moiety, wherein the linking moiety comprises an scFv or a nanobody. Another embodiment is the binding agent having a first linking moiety that comprises biotin. It is possible to use multiple linking moieties, for example without limitation, the binding agent can be a mouse antibody specific for a target antigen, where the antibody has an epitope recognized by a rabbit anti-mouse IgG antibody, where the rabbit antibody bears a biotin linking moiety, which in turn binds to streptavidin which is bound to a microbubble.

The binding agent and/or linking moieties can further comprise a cleavage site situated between the targeting moiety and the attachment to the microbubble (or site for such attachment), permitting convenient dissociation of the cell and microbubble after separation from the sample. For example, a biotin linking moiety can be connected to an antibody via a disulfide bond, which can be later cleaved using a reducing agent to release cells from microbubbles.

In embodiments wherein multiple different binding agents are used, each microbubble can have binding agents that target only one target antigen, or can have a mixture of binding agents that target two or more target antigens. In embodiments wherein the binding agent is indirectly bound to the microbubble, the different binding agents can use a common linking moiety. For example, the different binding agents can be biotinylated, so that all are bound to streptavidin-coated microbubbles. The binding agents can be used in equal concentrations, or in different concentrations.

### Microbubbles

Microbubbles of the disclosure are selected for size and buoyancy. For the present methods, microbubbles are composed of a rigid material, for example without limitation, glass, polystyrene, and the like. In an embodiment, the microbubbles are glass. In another embodiment, the microbubbles comprise polymers. In an embodiment, the microbubbles comprise polystyrene. In another embodiment, the microbubbles comprise a mixture of glass microbubbles and polymer microbubbles.

The microbubbles may be solid or hollow. Hollow microbubbles have a wall thickness sufficient to make the microbubble rigid and capable of resisting breakage or rupture when handled under reasonable laboratory conditions, for example under pressures of from about 0.2 to about 5 atm. In an embodiment, hollow microbubbles have an average wall thickness of from about 0.2 µm to 2.0 µm. In an embodiment, hollow microbubbles have an average wall thickness of from about 0.4 µm to 1.0 µm. In an embodiment, hollow microbubbles have an average wall thickness of from about 0.5 µm to 0.8 µm. In an embodiment, the microbubbles have an average wall thickness of about 0.7 µm. Hollow microbubbles can also comprise a gas, liquid, or vacuum interior. The gas can be hydrogen, helium, nitrogen, oxygen, neon, CO₂, and the like. Suitable gases can be selected that do not degrade the microbubble walls.

The microbubbles must have a density less than the density of the samples from which fetal cells will be enriched in order to be buoyant. In general, the microbubble must have a density low enough such that when bound to a fetal cell, the combination of microbubble and fetal cell together (including the binding agent) must be more buoyant than the non-fetal cells in the sample, to a degree sufficient to separate it from the non-fetal cells. The buoyancy cannot be too high, or the microbubbles will not remain suspended in the bulk sample long enough to interact with the fetal cells and/or binding agents. However, if the buoyancy is too low, the microbubbles will separate bound fetal cells from the bulk of the sample slowly, or fail to effect a complete separation. In an embodiment, the microbubbles have a density that is at least about 20%, 25%, 30%, 35%, 40%, 45%, 50%, 55%, 60%, 65%, 70%, 75%, or 80% of the density of the sample. In an embodiment, the microbubbles have a density that is no more than about 98%, 95%, 90%, 85%, 80%, 75%, 70%, 65%, 60%, 55%, or about 50% of the density of the sample. In an embodiment, the microbubbles have a density that is between about 40% and about 80% of the density of the sample. In an embodiment, the microbubbles have a density that is between about 50% and about 75% of the density of the sample. In an embodiment, the microbubbles have a density that is about 70% of the density of the sample. In an embodiment, the microbubbles have a density that is between about 0.20 g/cm³ and 0.90 g/cm³. In an embodiment, the density is about 0.20, 0.30, 0.40, 0.50, 0.60, 0.65, 0.70, 0.75, 0.80, 0.85, or 0.90 g/cm³. In an embodiment, the microbubbles have a density that is at least about 20%, 25%, 30%, 35%, 40%, 45%, 50%, 55%, 60%, 65%, 70%, 75%, or 80% of the density of the non-fetal cells in the sample. In an embodiment, the microbubbles have a density that is no more than about 98%, 95%, 90%, 85%, 80%, 75%, 70%, 65%, 60%, 55%, or about 50% of the density of the non-fetal cells in the sample. In an embodiment, the microbubbles have a density that is at least about 20%, 25%, 30%, 35%, 40%, 45%, 50%, 55%, 60%, 65%, 70%, 75%, or 80% of the density of sample medium (i.e., the fluid component of the sample, excluding fetal and non-fetal cells). In an embodiment, the microbubbles have a density that is no more than about 98%, 95%, 90%, 85%, 80%, 75%, 70%, 65%, 60%, 55%, or about 50% of the density of the sample medium.

Microbubbles are prepared using methods known in the art (see, for example, Morris, US 2016/0152513), or can be obtained from commercial sources (for example, 3M, Akadeum Life Sciences).

### Method of Enriching a Cell Population

Cell populations are enriched in fetal cells by the method of the disclosure. A biological sample is provided from a pregnant subject, and is contacted with one or more binding agents and microbubbles. The binding agents link fetal cells to microbubbles, and the microbubble buoyancy results in separation of the fetal cells from the non-fetal cells in the sample.

The pregnant subject biological sample can be any sample that contains or is likely to contain an adequate number of fetal cells, which can be obtained with no more than negligible risk to the subject or the pregnancy. Suitable biological samples include, without limitation, peripheral blood and cervical secretions.

Biological samples can be collected during any week of gestation, however, the incidence of fetal cells in the sample can vary with the week of gestation. In some embodiments, the biological sample is collected between about week 1 and about week 26. In some embodiments, the biological sample is collected between about week 3 and about week 20. In some embodiments, the biological sample is collected between about week 5 and about week 18. In some embodiments, the biological sample is collected between about week 6 and about week 15. In an embodiment, the biological sample is collected before about gestational week 17, 16, 15, 14, 13, 12, 11, 10, 9, 8, 7, 6, 5, or 4. In an embodiment, the biological sample is collected after about gestational week 1, 2, 3, 4, 5, 6, 7, 8, 9, or 10. In an embodiment, the biological sample is collected when the gestational age is between about 6 weeks and about 15 weeks.

Using the methods described herein, a sufficient number of fetal cells, in good condition, is reliably obtained from maternal samples taken during pregnancy. The number of fetal cells reliably obtained is at least about 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 80, or 100 fetal cells per sample. This number of fetal cells is sufficient to perform NIPD with the accuracy needed for and acceptable diagnostic assay. The number of cells obtained varies from subject to subject, but is considered reliable if at least that number of fetal cells is obtained from at least about 70, 75, 80, 85, 90, 95, 96, 97, 98, or 99 % of samples from the subjects tested.

### a) Blood

Blood samples are obtained using methods known in the art, such as methods used for molecular biology, paternity testing, HLA testing, and the like. In general, blood samples are drawn using a syringe or similar device, such as a Vacutainer® tube. The syringe or tube can contain anticoagulants, preservatives, stabilizers, fixatives, and the like to preserve the sample in a form suitable for analysis. In an embodiment, the syringe or tube contains an anticoagulant. In an embodiment, the syringe or tube contains EDTA (ethylenediaminetetraacetic acid). In an embodiment, the syringe or tube contains a fixative. In an embodiment, the syringe or tube contains paraformaldehyde. In an embodiment, the syringe or tube contains EDTA and paraformaldehyde.

The amount of blood drawn depends on the efficiency of the fetal cell enrichment process, the number of fetal cells in the blood or expected in the blood, and the number of fetal cells needed for the analysis described below. The number of fetal cells needed also depends on factors such as the number of fetuses present, maternal mosaicism, fetal mosaicism, placental mosaicism, and maternal hyperproliferative disorders such as cancer. In methods of the disclosure, the target number of fetal cells to be obtained is at least about 2, 3, 4, 5, 6, 7, 8, 9, or 10, or more fetal cells. The amount of blood drawn can be about 1 mL to about 40 mL. In an embodiment, the amount of blood drawn is about 1, 2, 5, 7, 8, 9, 10, 15, 20, 25, 30, 35, 36, 37, 38, 39, or 40 mL. In an embodiment, the amount is about 10 mL. In an embodiment, the amount is about 30 mL. In an embodiment, the amount is about 40 mL.

The sample is optionally fixed with a suitable fixative to preserve the cells. Suitable fixatives include formaldehyde (or paraformaldehyde), glutaraldehyde, mixtures thereof, and the like. The fixative is can be provided as a solution, for example in phosphate-buffered saline (PBS), and the like, or as a solid, for example spray-dried on the walls of a collecting container. The osmolarity of the solution used is generally sufficiently close to normal physiological osmolarity that cells are not ruptured prior to fixation. The amount of fixative used is an amount sufficient to fix the sample. In an embodiment, the fixative solution is 5% paraformaldehyde in PBS. In some embodiments, the amount of fixative solution is about 0.01, 0.05, 0.10, 0.15, 0.20, 0.25, 0.3, 0.35, 0.4, 0.45, 0.50, 0.55, 0.60, 0.65, 0.67, 0.70, 0.75, 0.80, 0.85, 0.90, 1.0, 1.25, 1.50, or 2.0 × the volume of the sample. In an embodiment, the amount is about 0.50 to about 0.75. In an embodiment, the amount is about 0.67 × the sample volume. In some embodiments, the cells are washed after fixation.

Normal blood contains about 4-6 × 10⁹ red blood cells (RBC) per mL, and about 4-6 × 10⁶/mL white blood cells. In contrast, the expected number of fetal cells in a maternal blood sample is about 1-5 cells/mL. Accordingly, the sample is optionally processed to remove RBCs. RBCs can be removed by standard methods, for example by binding to anti-glycophorin-A antibodies, which can further be bound to a solid support for separation, or can simply aggregate RBCs from suspension. RBCs can also be removed by selective lysis, for example using ammonium chloride or a hemolytic surfactant (see, e.g., M. Manaargadoo-Catin et al., Adv Colloid Interface Sci (2016) 228:1-16). In an embodiment, the RBCs are removed by contact with a hemolytic surfactant. In an embodiment, the surfactant is a polyoxyethylene surfactant. In an embodiment, the surfactant is Triton X-100®. In an embodiment, the surfactant further comprises a solution. In an embodiment, the surfactant is a polyoxyethylene surfactant solution or suspension. The concentration of the solution is selected to lyse maternal RBCs without affecting fetal cells. In an embodiment, the maternal RBCs are lysed in a mixture comprising a polyoxyethylene surfactant wherein the surfactant is present at a concentration of about 0.0001% to about 0.1% w/w. In an embodiment, the surfactant concentration is about 0.001% to about 0.05% w/w. In an embodiment, the surfactant concentration is about 0.01% to about 0.05% w/w.

Following pre-treatment to reduce or eliminate the maternal RBCs, the remaining sample is washed to remove or reduce the amount of surfactant and fixative that may remain. Conventional methods for molecular biology can be employed. For example, without limitation, the sample can be diluted in PBS, which may optionally contain bovine serum albumin (BSA), and then centrifuged. The resulting pellet can be further washed, and the supernatants removed by aspiration.

Whole Blood Protocol: This exemplary procedure is performed to provide a population of cells from whole blood (without removing RBCs) that is substantially enriched in trophoblasts, and optionally stained to distinguish between trophoblasts and maternal cells in the sample.

Maternal blood samples are collected in several (for example, about 2 to about 5) 10 mL EDTA Vacutainer® tubes for trophoblast cell enrichment, and optionally an additional tube (for example, a 4 mL tube) for extraction of maternal genomic DNA (gDNA) and fetal cfDNA for fetal sex determination. Paternal samples are optionally collected (saliva in 2 mL EDTA). Control samples from healthy, non-pregnant individuals can be collected for lymphoblast and other cell spike-in experiments, or as controls.

Maternal gDNA, and optionally paternal gDNA, is extracted from whole blood by any convenient method, for example using magnetic beads. Fetal cfDNA can be extracted from maternal plasma on the same platform, for example, using a large volume MagNA Pure compact nucleic acid isolation kit I (Roche). This cfDNA can be used in a Y-chromosome qPCR reaction to determine the fetal gender, based on the detection of amplicons for DYS14 and SRY (see, e.g., A.M. Breman et al., Prenat Diagnos (2016) 36:1009-19; L. Vossaert et al., Prenat Diagnos (2018) 38:1069-78).

The blood sample is processed in aliquots of 2, 5, 10, 20 or more mL, placed in plastic conical tubes of, for example, about 5, 15, or 50 mL. The blood can be treated unfixed, or a mild paraformaldehyde fixation may be performed. A separation buffer (for example, PBS with 2 mM EDTA, 0.5% BSA, 0.09% sodium azide, pH 7.2; free of Ca²⁺, Mg²⁺, and biotin) is added to each tube in a volume equal to about 1% to about 100% of the blood volume in the tube. In some embodiments, the volume of separation buffer added is about 1, 2, 3, 4, 5, 6, 7, 8, 9, 10, 12, 15, 20, 25, 30, 35, 40, 45, 50, 55, 60, 70, 80, 90, or 100% of the blood volume in the tube.

About one to about 5 different binding agents (for example, antibodies) are selected for binding to fetal cell antigens (for example, the trophoblast surface), targeting antigens such as, for example, HLA-E, HLA-G, MCAM (CD146), ATG9B, EpCAM, TROP-2, CD31, CD141, CD144, MMP9, ITGA1, CSHI, CD105, LVRN, EGFR, ErbB2, ErbB3, ErbB4, or annexin A4. The concentration of each antibody is can be from about 0.1 to about 10 µg per 5 mL of whole blood, typically about 1.0 µg per 5 mL whole blood. In some embodiments, the concentration of each antibody is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 7.0, 8.0, 9.0, or about 10.0 µg per 5 mL whole blood. These binding agents include a linking moiety capable of binding to microbubbles (or another linking moiety(ies) attached to the microbubbles), for example biotin. In some embodiments, the binding agent is a biotinylated antibody, and a linking moiety (for example, avidin, streptavidin, monomeric avidin) is attached to the microbubbles. In some embodiments, the binding agent is a biotinylated antibody, and a linking moiety (for example, avidin, streptavidin, monomeric avidin) is attached to a second antibody, wherein the second antibody binds to or is capable of binding to the microbubbles. In other embodiments, the binding agent is a labeled antibody, and a second antibody specific for the binding agent is bound to the microbubbles or is capable of binding to the microbubbles. In some embodiments, the second antibody is biotinylated, and the microbubbles are coated with streptavidin. The binding agents can optionally be labeled, for example conjugated with a dye such as FITC (fluorescein isothiocyanate), or can be unlabeled. In some embodiments, the binding agents are murine antibodies, and the second antibodies are caprine anti-mouse antibodies. In some embodiments, the anti-mouse antibodies are biotinylated. In some embodiments, the secondary antibodies are added before cells are contacted with the microbubbles. The antibody-blood mixture is incubated for about 5 to about 60 min at about 4° C. In some embodiments, the mixture is incubated for about 20 min at 4° C. For nuclear staining, a dye such as Hoechst dye can be added if blood is unfixed, or DAPI can be added if blood was fixed.

Additional separation buffer (for example without limitation, Akadeum separation buffer) is added to each tube, and the mixture is centrifuged. The amount of buffer added is a volume equal to about 1% to about 200% of the sample volume in the tube. In some embodiments, the volume of separation buffer added is about 1, 2, 3, 4, 5, 6, 7, 8, 9, 10, 12, 15, 20, 25, 30, 35, 40, 45, 50, 55, 60, 70, 80, 90, 100, 120, 140, 150, 160, 170, 180, 190, or about 200% of the sample volume in the tube. In some embodiments, the amount added is about 50% of the sample volume. The mixture is centrifuged for about 1 to about 20 min, at about 500 to about 1,000 × g. In some embodiments, the mixture is centrifuged for about 1, 2, 3, 4, 5, 6, 7, 8, 9, 10, 11, 12, 13, 14, 15, 16, 17, 18, 19, or 20 min. in some embodiments, the mixture is centrifuged at about 500, 600, 700, 800, 900, or 1,000 × g. In some embodiments, the mixture is centrifuged for about 5 min at about 700 × g).

After centrifugation, the supernatant is aspirated, and separation buffer (for example, the separation buffer set forth above) is added in a volume equal to about 1% to about 100% of the original blood volume. In some embodiments, the volume of separation buffer added is about 1, 2, 3, 4, 5, 6, 7, 8, 9, 10, 12, 15, 20, 25, 30, 35, 40, 45, 50, 55, 60, 70, 80, 90, or 100% of the original blood volume. In some embodiments, the amount added is about 50% of the original blood volume. If using a binding agent that is not biotinylated, the secondary antibody (for example, a biotinylated anti-mouse antibody, or other antibody specific for the binding agent used) is added, and the sample is again incubated, washed, and resuspended as above.

Streptavidin-coated microbubbles (Akadeum, #32211-120) are suspended by vigorous mixing until the mixture appears homogenous, then immediately added to the samples. About 0.5 µL of microbubbles is added per mL of starting whole blood. A pipette is set to a volume equal to one half the volume of the sample-microbubble mixture, and the mixture is gently mixed by trituration using a 1000 µL or larger low retention tip for about 30 strokes. Separation buffer (about 1.5 mL per starting mL of whole blood) is then added to each sample, and the samples are centrifuged (about 5 min, at about 400 × g, at about 4° C.). The white microbubble layer is then aspirated off, to provide a cell population enriched in fetal cells as a composition of fetal cell-binding agent-microbubble complexes. Alternatively, the RareCyte Accucyte® device can be used to express the microbubbles from the top of the tube, collecting them in a microfuge tube separated from the non-fetal cells in the separation tube. The fetal cell-binding agent-microbubble complexes can be spread on a slides for individual cell picking of cell-bubble units on the RareCyte CyteFinder. Alternatively the cells can be released from the microbubble complexes, for example without limitation by incubation with papain, to form a fetal cell-enriched cell population. The released fetal cells can further be applied to a microfluidic cell separator (for example, using a Namocell Namo™) to deposit individual cells in microtiter wells.

One option is to process all nucleated cells for downstream whole genome amplification and sequencing. Alternatively, the fetal cells can be stained with fetal cell-specific antibodies conjugated to a dye, for example, FITC. One option is to use a labeled antibody in combination prior to the microbubble capture. Another option is to stain the fetal cells with an FITC-conjugated antibody after capture by microbubbles. For example, one can capture with biotinylated anti-EpCAM, and stain with FITC-anti-CD105 or FITC-anti-HLA-G. Cells can also be stained with PE-anti-CD45, with selection of only cells that are positive for FITC and the selected target antigen. With this staining, one can pick with RareCyte, or sort in a microfluidic device, only FITC-positive cells. The cells are then processed using whole genome amplification and NGS.

### b) Cervical Secretions

Other suitable biological samples can be used instead of maternal blood. In particular, cervical secretions can be collected by using an endocervical brush and other methods (see, e.g., A.N. Imudia et al., Fertil Steril (2010) 96(6): 1725-30; G. Moser et al., Hum Repro Update (2018) 24(4):484-96; C.V. Jain et al., Sci Transl Med (2016) 8:363re4). Cervical secretion samples also contain maternal cells such as squamous cells, and may contain additional debris, such as blood elements, spermatozoa, mucus, and particulate contaminants. Mucus can be lysed using known agents, for example mucolytic agents like L-acetyl cysteine, and/or enzymes such as liberase blendzyme (see, e.g., M.G. Katz-Jaffe et al., BJOG (2005) 112:595-600). As with blood samples as described above, cervical secretion samples can also be washed using conventional methods for molecular biology. For example, without limitation, the sample can be diluted in PBS, which may optionally contain bovine serum albumin (BSA), and then centrifuged. The resulting pellet can be further washed, and the supernatants removed by aspiration.

### c) Cell Population Enrichment

Whether obtained from a maternal blood sample, a cervical secretion sample, or another biological sample obtained from a pregnant subject, the biological sample is then enriched in fetal cells (fnRBCs and/or trophoblasts) following the pre-treatment described above. Enrichment results in a population of cells in which the fetal cells are present in a ratio of fetal cell to other cells of at least about 1:100, at least about 1:80, at least about 1:60, at least about 1:50, at least about 1:40, at least about 1:30, at least about 1:20, at least about 1:10, at least about 1:5, about 1:1, greater than about 1:1, greater than about 5:1, greater than about 10:1, or greater than about 100:1. In an embodiment, the fetal cell:other cell ratio is between about 1:1,000 and about 100:1. In an embodiment, the fetal cell:other cell ratio is between about 1:100 and about 50:1. In an embodiment, the fetal cell:other cell ratio is between about 1:10 and about 10:1. In an embodiment, the fetal cell:other cell ratio is about 1:1.

Cell population enrichment in a sample is accomplished in the methods of the disclosure by contacting the sample with a binding agent that comprises a targeting moiety specific for a fetal cell antigen, and a buoyant microbubble, wherein the binding agent binds to the microbubble surface. The binding agent, as described above, may be bound to the microbubble either before, after, or simultaneously with binding to the fetal cell antigen. Multiple different binding agents may be used together.

The sample is contacted with the binding agent(s) for a period of time sufficient to permit specific binding, forming fetal cell-binding agent complexes, and may employ conventional molecular biology techniques. For example, a mixture of two or more different binding agents, such as biotinylated antibodies, can be added to a sample and incubated at 4° C. for about 1 to about 5 hours on a laboratory shaker. In an embodiment, a mixture of 2 to 10 different binding agents is used. In an embodiment, a mixture of 3 to 7 different binding agents is used. In an embodiment, a mixture of about 3 to about 5 different binding agents is used. In an embodiment, the binding agents comprise antibodies specific for HLA-G, TROP-2, and EpCAM. In an embodiment, additional elements are added to the sample to aid in separation, for example without limitation PBS, EDTA, BSA, sodium azide, and the like. The cells can be centrifuged and resuspended to remove any excess binding agent, for example, by centrifugation at 400 × g.

Microbubbles can be bound to binding agents before, after, or simultaneously with the fetal cells. For example, streptavidin-coated microbubbles can be added to PBS and suspended by vigorous shaking. This suspension can then be added to a suspension of fetal cell-binding agent complexes, mixed, and incubated, to form fetal cell-binding agent-microbubble complexes. Mixing can be accomplished by stirring, shaking, passage through a mixing manifold, trituration, and other low shear methods. For example, the mixture can be gently mixed by trituration using a 1000 µL low retention pipette tip for about 30 strokes. Incubation is conducted at a temperature and a period of time sufficient for binding agents and microbubbles to bind each other. In the case of biotin and streptavidin, little time is required.

The fetal cell-binding agent-microbubble complexes are then separated from other cells and components of the biological sample due to the microbubble buoyancy. This can be accomplished by letting the sample stand, or can be accelerated by centrifugation. The fetal cell-binding agent-microbubble complexes float to the top of the sample, where they are removed and separated from the sample by aspiration or other techniques to provide a cell population that is enriched in fetal cells, as set forth herein.

The fetal cells may optionally be stained for ease of visualization and identification, for example using standard microbiology and molecular biology techniques. Fetal cells can be permeabilized and contacted with, for example, DAPI and/or labeled antibodies specific for cytokeratin, EpCAM, and other fetal cell antigens as described herein. In an embodiment, the sample is stained with DAPI, anti-cytokeratin, and anti-CD45 (anti-CD45 serving as a marker for maternal leukocytes).

### Nucleic Acids

The fetal cell-binding agent-microbubble complexes can be separated into aliquots having about 1 to about 5 cells, by dilution into multiple wells, or can be mechanically picked using instruments such as, for example, a CytePicker® retrieval module (RareCyte, Inc., Seattle, WA). In an embodiment, the complexes are separated by dilution into wells containing 0, 1, or 2 cells. In an embodiment, the complexes are separated by dilution into wells containing 0 or 1 cells. The single fetal cell complexes may optionally be dissociated to provide free fetal cells. Alternatively, the complexes can be separated using a microfluidic device, for example without limitation, a Namocell Namo™, Hana™ (Namocell Inc., Mountain View, CA), or WOLF/N1 (Nanocellect Biomedical, Inc., San Diego, CA) single cell sorter and dispensers.

If desired, the cells can be dissociated from the microbubbles before or after the separation. The dissociation method employed will generally depend on the nature of the binding agents employed. For example, where the binding agent is an antibody, it can be removed by changing the pH, or competition with a protein or peptide that is bound by the antibody. Alternatively, antibodies can be cleaved with known enzymes, such as for example, pepsin, papain, FabRICATOR®, FabALACTICA® (Genovis Inc., Cambridge, MA), and similar enzymes in order to release the cell from the microbubble complex. Alternatively, where the binding agent comprises a cleavable linker, the linker can be cleaved to release the cell. For example, where the binding agent comprises two or more fragments joined by one or more disulfide bonds, the cell may be released by reducing the disulfide bonds. Alternatively, the cell can be released from the complex by cleaving the fetal cell antigen, for example, cleaving HLA-G using MMP-2 (see, e.g., R. Rizzo et al., Mol Cell Biochem (2013) 381:243-55) or cleaving TROP-2 using TACE (TNFα converting enzyme) (Y. Mori et al., J Biol Chem (2019) 294:11513-24; T. Stoyanova et al., Genes Dev (2012) 26(20):2271-85).

Nucleic acids are obtained from a cell population enriched in fetal cells by lysis. In an embodiment, the fetal cells are lysed in a pool comprising no more than about 50, 40, 30, 25, 20, 15, 10, 9, 8, 7, 6, 5, 4, 3, 2 or 1 cells. In an embodiment, the fetal cells are lysed in individual pools. In an embodiment, multiple fetal cells are lysed in the same pool if they were isolated as a clump of from about 2 to about 20 fetal cells. Alternatively, the cell population can be sorted by known techniques such as FACS (fluorescence activating cell sorting) or using a microfluidic device, for example without limitation, a Namocell Namo™ or Hana™ single cell dispenser (Namocell Inc., Mountain View, CA) or WOLF/N1 microfluidic cell sorter/dispenser (Nanocellect Biomedical, Inc., San Diego, CA), or submitted directly into a sorting / labeling input device for single-cell sequencing.

### Genotyping

Nucleic acids obtained by the methods herein are examined for indications of a genetic difference. All nucleic acids, or only subsets, can be examined, for example, one can examine genomic DNA (gDNA), messenger RNA (mRNA), or both. In some embodiments, the nucleic acids are amplified before examination. In an embodiment, gDNA is amplified by whole genome amplification (WGA) prior to examination.

Indications of a genetic difference can take several different forms: in general, the genetic difference is a difference between the nucleic acids in the biological sample, and the sequence, organization, gene or fragment copy number, and the like of a consensus healthy singleton fetus. For example, without limitation, the difference can be a copy number variation (CNV) such as the duplication or absence of an entire chromosome (aneuploidy), the duplication or absence of a part of a chromosome (partial aneuploidy), and the duplication or absence of one or more genes or parts of genes. The difference can also be a polymorphism, for example without limitation, a translocation between two chromosomes, an indel (insertion or deletion), or a single nucleotide variant (SNV) Differences further include the presence of two or more sets of chromosomes, indicating multiple fetuses (e.g., twins, triplets, and the like). Differences also include genotypes wherein a pair of chromosomes consists of two identical or partially identical copies, which can indicate uniparental disomy (see, e.g., K, Yauy et al., Genet Med (2019) doi.org/ 1.1038/s41436-019-0704-x). Differences further include mosaicism, such as confined placental mosaicism, and alleles that are associated with pathological conditions or genetic disorders.

A number of heritable genetic disorders are known. An embodiment is the method wherein the pathological condition is 1p36 deletion syndrome, 18 p deletion syndrome, 21-hydroxylase deficiency, Alpha 1-antitrypsin deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Aicardi-Goutieres syndrome, Albinism, Alexander disease, alkaptonuria, Alport syndrome, Alternating hemiplegia of childhood, Amyotrophic lateral sclerosis - Frontotemporal dementia, Alström syndrome, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Björnstad syndrome, Bloom syndrome, Birt-Hogg-Dube syndrome, Brody myopathy, Brunner syndrome, CADASIL syndrome, CARASIL syndrome, Chronic granulomatous disorder, Campomelic dysplasia, Canavan disease, Carpenter Syndrome, Cerebral dysgenesis-neuropathy-ichthyosis-keratoderma syndrome (SEDNIK), Cystic fibrosis, Charcot-Marie-Tooth disease, CHARGE syndrome, Chediak-Higashi syndrome, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn’s disease, Crouzon syndrome, Crouzonodermoskeletal syndrome (Crouzon syndrome with acanthosis nigricans), Darier’s disease, Dent’s disease (Genetic hypercalciuria), Denys-Drash syndrome, De Grouchy syndrome, Down Syndrome, Di George’s syndrome, Distal hereditary motor neuropathies, multiple types, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich’s ataxia, G6PD deficiency, Galactosemia, Gaucher disease, Gerstmann-Sträussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington’s disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, familial, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer’s syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimeta-physeal dysplasia, Strudwick type), Tay-Sachs disease, Tetrahydrobiopterin deficiency, Thanatophoric dysplasia, Treacher Collins syndrome, Trisomy 8, Trisomy 9, Trisomy, 22, Tuberous sclerosis complex (TSC), Turner syndrome, Usher syndrome, Variegate porphyria, von Hippel-Lindau disease, Waardenburg syndrome, Weissenbacher-Zweymüller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (triple X syndrome), XXXX syndrome (48, XXXX), XXXXX syndrome (49, XXXXX), XYY syndrome (47,XYY), or Zellweger syndrome. An embodiment is the method wherein the pathological condition is Angelman syndrome, Canavan disease, Charcot-Marie-Tooth disease, Cri du chat syndrome, Cystic fibrosis, DiGeorge syndrome, Down syndrome, Duchenne muscular dystrophy, Familial hypercholesterolemia, Haemochromatosis, Hemophilia, Klinefelter syndrome, Neurofibromatosis, Phenylketonuria, Polycystic kidney disease (PKD1 or PKD2, Prader-Willi syndrome, Sickle cell disease, Spinal muscular atrophy, Tay-Sachs disease, or Turner syndrome.

The nucleic acids can be examined by methods known in the art for comparing nucleic acid sequences and/or quantities. In some embodiments, the method is quantitative polymerase chain reaction amplification (qPCR), array comparative genomic hybridization (array CGH), or next generation sequencing (NGS).

Commercially available sequencing equipment can be used for genotyping, for example without limitation, the Illumina, ThermoFisher (Ion Torrent™), Oxford Nanopore Technologies, Pacific Biosciences, and Qiagen platforms.

It will be apparent to those skilled in the art that a number of different sequencing methods and variations can be used. One method that can be used involves paired end sequencing. Fluorescently labeled sequencing primers can be used to simultaneously sequence both strands of a dsDNA template, as described e.g., by S. Wiemann et al., Anal Biochem (1995) 224:117-21; S. Wiemann et al., Anal Biochem (1996) 234:166-74. This technique has demonstrated multiplex co-sequencing using the four-color dye terminator reaction chemistry pioneered by Prober et al., Science (1987) 238:336.

Comparative Genome Hybridization (CGH) is based on a quantitative two-color fluorescence in situ hybridization (FISH) on metaphase chromosomes. In this method, a test DNA (for example, DNA extracted from a trophoblast) is labeled in one color (for example, green) and mixed in a 1:1 ratio with a reference DNA (e.g., DNA extracted from a control cell) which is labeled in a different color (e.g., red), and the fluorescence is measured. Briefly, genomic DNA is amplified using a degenerate oligonucleotide primer (see for example, D. Wells et al., Nucleic Acids Res (1999) 27:1214-8), and the amplified DNA is labeled using, for example without limitation, Spectrum Green-dUTP (for the test DNA) or Spectrum Red-dUTP (for the reference DNA). The mixture of labeled DNA samples is precipitated with Cot1 DNA (Gibco-BRL) and resuspended in a hybridization mixture containing, for example, 50% formamide, 2 × SSC, pH 7, and 10% dextran sulfate. Prior to hybridization, the labeled DNA samples (i.e., the probes) are denatured for 10 minutes at 75° C., and allowed to cool at room temperature for 2 minutes. Likewise, the metaphase chromosome spreads are denatured using standard protocols (e.g., dehydration in ethanol, denaturation for 5 minutes at 75° C. in 70% formamide and 2 × SSC). Hybridization conditions include incubation at 37° C. for 25-30 hours in a humidified chamber, following by washes in 2 × SSC and dehydration using an ethanol series (see, e.g., D. Wells et al., Fertil Steril (2002) 78:543-49). The hybridization signal is detected using a fluorescence microscope, and the ratio of the green-to-red fluorescence can be determined using e.g., the Applied Imaging (Santa Clara, Calif.) computer software. If both genomes are equally represented in the metaphase chromosomes (i.e., no deletions, duplication or insertions in the DNA derived from the trophoblast cell), the labeling on the metaphase chromosomes is yellow or orange. Regions which are either deleted or duplicated in the trophoblast cell are stained red or green respectively.

DNA array-based comparative genomic hybridization (CGH-array) is a modified version of CGH and is based on the hybridization of a 1:1 mixture of the test and reference DNA probes on an array containing chromosome-specific DNA libraries (D.G. Hu et al., Mol Hum Reprod (2004) 10:283-89). Methods of preparing chromosome-specific DNA libraries are known in the art (see, for example, A. Bolzer et al., Cytogenet Cell Genet (1999) 84:233-40). Briefly, single chromosomes are obtained by microdissection or flow-sorting, and the genomic DNA of each of the isolated chromosomes is PCR-amplified using a degenerate oligonucleotide primer. To remove repetitive DNA sequences, the amplified DNA is subjected to affinity chromatography in combination with negative subtraction hybridization (using, for example, human Cot-1 DNA or centromere-specific repetitive sequence as subtractors) (see, e.g., J.M. Craig et al., Hum Genet (1997) 100:472-76). Amplified chromosome-specific DNA libraries are then attached to a solid support, for example, SuperAmine slides (TeleChem, USA), dried, baked and washed according to manufacturer is recommendation. Labeled genomic DNA probes (1:1 mixture of the test and reference DNAs) are mixed with non-specific carrier DNA (e.g., human Cot-1 and/or salmon sperm DNA, Gibco-BRL), ethanol-precipitated, and re-suspended in an hybridization buffer such as 50% deionized formamide, 2 × SSC, 0.1% SDS, 10% dextran sulphate and 5 × Denhardt’s solution. The DNA probes are then denatured at 80° C. for 10 minutes, pre-annealed at 37° C. for 80 minutes, and applied on the array for hybridization of 15-20 hours in a humid incubator. Following hybridization, the arrays are washed twice for 10 minutes in 50 % formamide/2 × SSC at 45° C. and once for 10 minutes in 1 × SSC at room temperature, following which the arrays are rinsed three times in 18.2 MΩ deionized water. The arrays are then scanned using a suitable fluorescence scanner, such as the GenePix 4000B microarray reader (Axon Instruments, USA), and analyzed using the GenePix Pro. 4.0.1.12 software (Axon).

NGS sequencing results are analyzed using techniques and software tools known in the art. For example, sequence alignments can be obtained using algorithms such as BWA-MEM in Burrows-Wheeler Aligner. Coverage counts can be obtained using software such as bedtools. Data can also be analyzed using software such as NxClinical (BioDiscovery, El Segundo, CA).

### Systems

Systems of the disclosure provide reagents suitable for the practice of the methods described herein. Systems comprise a binding agent having a targeting moiety specific for a fetal cell antigen, and a buoyant microbubble, wherein the binding agent binds to the microbubble surface, wherein the binding agent and microbubble form fetal cell-binding agent-microbubble complexes, wherein the complexes have an average ratio of fetal cell to microbubble of about 1:5 to about 5:1, and wherein the microbubble has a density of about 0.4 g/cm³ and about 0.8 g/cm³.

In some embodiments, the system further comprises a stain or a label for visualizing and/or identifying fetal cells. In some embodiments, the stain is DAPI. In some embodiments, the label is an antibody or other binding agent having a detectable label, wherein the antibody binds to the fetal cell.

In some embodiments, the microbubble has a diameter between about 10 µm and about 20 µm. In some embodiments, the microbubble has a diameter between about 13 µm and about 19 µm. In some embodiments, the microbubble has a diameter between about 16 µm and about 18 µm.

### Compositions

An aspect of the disclosure is a composition comprising a fetal cell-binding agent-microbubble complex and maternal cells, wherein the ratio of fetal cells to maternal cells is from about 1:1,000 to about 1,000:1. Compositions are useful for the prenatal diagnosis of genetic disorders, and as a source of fetal nucleic acids that can be used in non-invasive prenatal diagnostics. In an embodiment, the ratio of fetal cells to maternal cells is from about 1:100 to about 5:1. In an embodiment, the ratio is from about 1:10 to about 1:1. In embodiments, the fetal cell is a circulating trophoblast or a fetal nucleated red blood cell. In some embodiments, the ratio of fetal cell to microbubble is about 5:1 to about 1:5. In some embodiments, the ratio of fetal cell to microbubble is about 1:1. In some embodiments, the fetal cells are stained or labeled.

Another aspect is a fetal cell-enriched population of cells, wherein the ratio of fetal cells to maternal cells is from about 1:1,000 to about 1,000:1. In an embodiment, the ratio of fetal cells to maternal cells is from about 1:100 to about 5:1. In an embodiment, the ratio is from about 1:10 to about 1:1. In embodiments, the fetal cell is a circulating trophoblast or a fetal nucleated red blood cell. In some embodiments, the ratio of fetal cell to microbubble is about 5:1 to about 1:5. In some embodiments, the ratio of fetal cell to microbubble is about 1:1. In some embodiments, the fetal cells are stained or labeled.

Another aspect is the composition or the fetal cell-enriched cell population which is obtained by a method of the disclosure.

Another aspect is the use of a system or a composition or a fetal cell-enriched cell population of the disclosure for the diagnosis of a genetic disorder.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature, such as J. Sambrook & D.W. Russell, (2012) Molecular Cloning: A Laboratory Manual (4th ed.) Cold Spring Harbor, NY: Cold Spring Harbor Laboratory and J. Sambrook & D.W. Russell, (2001) Molecular Cloning: A Laboratory Manual (3rd ed.) Cold Spring Harbor, NY: Cold Spring Harbor Laboratory (jointly referred to herein as “Sambrook”); F.M. Ausubel (1987) Current Protocols in Molecular Biology, New York, NY: Wiley (including supplements through 2014); D.M. Bollag et al., (1996) Protein Methods, New York, NY: Wiley-Liss; L. Huang et al., (2005) Nonviral Vectors for Gene Therapy, San Diego, CA: Academic Press; M.G. Kaplitt et al., (1995) Viral Vectors: Gene Therapy and Neuroscience Applications, San Diego, CA: Academic Press; I. Lefkovits (1997) The Immunology Methods Manual: The Comprehensive Sourcebook of Techniques, San Diego, CA: Academic Press; A. Doyle et al., (1998) Cell and Tissue Culture: Laboratory Procedures in Biotechnology, New York, NY: Wiley; K.B. Mullis et al., (1994). PCR: The Polymerase Chain Reaction. Boston, MA: Birkhaüser; E.A. Greenfield (2014) Antibodies: A Laboratory Manual (2nd ed.), New York, NY: Cold Spring Harbor Laboratory Press; S.L. Beaucage et al., (2000) Current Protocols in Nucleic Acid Chemistry, New York, NY: Wiley, (including supplements through 2014); and S.C. Makrides, (2003) Gene Transfer and Expression in Mammalian Cells, Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### Trophoblast Enrichment From Maternal Blood Using RBC Lysis and Microbubble Separation

This procedure is performed to provide a population of cells that are substantially enriched in trophoblasts, and optionally stained to distinguish between trophoblasts and maternal cells in the sample.

(A) Maternal blood samples are collected in four 10 mL EDTA Vacutainer® tubes for trophoblast cell enrichment, and one 4 mL tube for extraction of maternal genomic DNA (gDNA) and fetal cfDNA for fetal sex determination. Paternal samples are optionally collected (saliva or 2 mL EDTA). Control samples from healthy, non-pregnant individuals are collected for lymphoblast and other cell spike-in experiments.

(B) Maternal gDNA, and optionally paternal gDNA, is extracted from whole blood on a MagNA Pure platform (Roche) using a MagNA Pure compact nucleic acid isolation kit I. Genomic DNA is extracted from paternal saliva using a MagNA Pure compact nucleic acid isolation kit I, large volume, on the same platform. Fetal cfDNA is extracted from maternal plasma on the same platform, using the large volume kit. The cfDNA is used in a Y-chromosome qPCR reaction to determine the fetal gender, based on the detection of amplicons for DYS14 and SRY (see, e.g., A.M. Breman et al., (2016); L. Vossaert et al., (2018)).

(C) The blood sample is fixed by addition of 0.67× volume of 5% paraformaldehyde in PBS for 10 minutes. Subsequently, the red blood cells are lysed by incubating for 8 minutes with 10× the original blood volume of RBC lysis buffer containing 0.12% TritonX-1009 in PBS. These two initial incubations are conducted at room temperature on a tube roller (20 rolls/minute). Then, 5× original blood volume of PBS containing 2% BSA is added. The sample is centrifuged (700 × g, 15 min, 4° C.) in two 500 mL tubes, and the supernatant is aspirated with vacuum to waste down to a volume of 5 mL per tube. The remaining cell pellets are combined, undergo two washing steps with PBS, and are reduced to 1 mL in PBS.

(D) A cocktail of three biotinylated enrichment antibodies containing 4 µg of each antibody is added to each sample, and incubated for two hours at 4° C. on a laboratory shaker. The three commercial enrichment antibodies are biotinylated mouse anti-human HLA-G, biotinylated mouse anti-human TROP-2 (both Novus Biologicals), and biotinylated mouse anti-human EpCAM (BioLegend). Separation buffer (1 mL: PBS with 2 mM EDTA, 0.5% BSA, 0.09% sodium azide, pH 7.2, free of Ca²⁺, Mg²⁺, and biotin) is added to each sample, and the samples centrifuged at 400 × g. The pellet is resuspended at 10⁷ cells per 50 µL of separation buffer.

(E) Streptavidin-coated microbubbles (5 µL, Akadeum, #32211-120) are suspended by vigorous mixing until the mixture appears homogenous, then immediately added to the samples. A pipette is set to a volume equal to one half the volume of the sample-microbubble mixture, and the mixture is gently mixed by trituration using a 1000 µL low retention tip for about 30 strokes. Separation buffer (3 mL) is then added to each sample, and the samples are centrifuged (5 min, 400 × g, 4° C.). The white microbubble layer is then aspirated off, to provide a cell population enriched in fetal cells. Alternatively, the RareCyte Accucyte® device can be used to express the microbubbles from the top of the tube, collecting them in a microfuge tube separated from all other cells in the separation tube.

(F) The trophoblast cell population is optionally centrifuged, resuspended, and treated with permeabilization solution (720 µL, BD Bioscience #554723), stain reagent (250 µL, anti-cytokeratin, anti-CD45) and DAPI nuclear stain (10 µL). The resulting mixture is incubated for 1 hour at ambient temperature, flicking the tubes every 15 minutes, to provide an enriched cell population in which trophoblasts and maternal cells are labeled with contrasting stains.

(G) After immunostaining, the enriched cell population is spread into CyteSlides (RareCyte) in PBS, and subsequently scanned using a CyteScanner or CyteFinder® (RareCyte). All identified trophoblast candidates are then curated manually, based on positive cytokeratin staining and its pattern, negative for CD45, and nuclear morphology. Using the CytePicker® module (RareCyte), all putative trophoblasts are picked individually with a 40 µm diameter needle and deposited into 200 µL PCR tubes in 2 µL PBS to provide individual, isolated fetal cells. All cells are stored at -80° C. until further processing.

(H) Alternatively, instead of proceeding as set forth in part (G) above, the enriched cell population is applied to a Namocell Namo™ single cell sorter, and is partitioned into single cell samples and stored at -80° C. until further processing.

### Example 2

### Trophoblast Enrichment From Maternal Blood by Adding Antibodies and Microbubbles to Whole Blood

This procedure is performed to provide a population of cells from whole blood that are substantially enriched in trophoblasts, and optionally stained to distinguish between trophoblasts and maternal cells in the sample.

(A) Maternal blood samples are collected in four 10 mL EDTA Vacutainer® tubes for trophoblast cell enrichment, and one 4 mL tube for extraction of maternal genomic DNA (gDNA) and fetal cfDNA for fetal sex determination. Paternal samples are optionally collected (saliva or 2 mL EDTA). Control samples from healthy, non-pregnant individuals are collected for lymphoblast and other cell spike-in experiments.

(B) Maternal gDNA, and optionally paternal gDNA, is extracted from whole blood on a MagNA Pure platform (Roche) using a MagNA Pure compact nucleic acid isolation kit I. If saliva from the father is used, gDNA is extracted using a MagNA Pure compact nucleic acid isolation kit I, large volume, on the same platform. Fetal cfDNA is extracted from maternal plasma on the same platform, using the large volume kit. This cfDNA is used in a Y-chromosome qPCR reaction to determine the fetal gender, based on the detection of two amplicons for DYS14 and SRY (see, e.g., A.M. Breman et al., (2016); L. Vossaert et al., (2018)).

(C) The blood sample is processed in aliquots of 10 mL, placed in plastic conical tubes of 15 or 50 mL. The blood can be treated unfixed, or a mild paraformaldehyde fixation may be performed. One tenth volume of separation buffer or similar is added to the tube.

Three to five antibodies for binding to the trophoblast surface are selected from HLA-E, HLA-G, MCAM (CD 146), ATG9B, EpCAM, TROP-2, CD31, CD141, CD 144, MMP9, ITGA1, CSHI, CD105, LVRN, EGFR, ErbB2, ErbB3, ErbB4, or annexin A4 antibodies. The antibodies are biotinylated, and used at a concentration of each antibody of about 1 µg antibody per 5 mL of whole blood. The antibody-blood mixture is incubated for 20 min at 4° C.

Separation buffer (PBS with 2 mM EDTA, 0.5% BSA, 0.09% sodium azide, pH 7.2; free of Ca²⁺, Mg²⁺, and biotin; 0.5 × the original blood volume) is added, and the mixture is centrifuged for 5 min at 700 × g (4° C.). The supernatant is aspirated, and additional separation buffer (0.5 × the original blood volume) is added.

(D) Streptavidin-coated microbubbles (Akadeum, #32211-120) are suspended by vigorous mixing until the mixture appears homogenous, then immediately added to the samples. About 0.5 µL of microbubbles is added per mL of starting whole blood. A pipette is set to a volume equal to one half the volume of the sample-microbubble mixture, and the mixture is gently mixed by trituration using a 1000 µL or larger low retention tip for about 30 strokes. Separation buffer (1.5 mL per mL starting whole blood) is then added to each sample, and the samples are centrifuged (5 min, 400 × g, 4° C.). The white microbubble layer is then aspirated off, to provide a cell population enriched in fetal cells. Alternatively, the RareCyte Accucyte® device can be used to express the microbubbles from the top of the tube, collecting them in a microfuge tube separated from all other cells in the separation tube. The microbubbles can be spread on slides for individual cell picking of cell-bubble units on the RareCyte CyteFinder®. Alternatively the cells can be released from the microbubbles by incubation with papain and the released cells applied to the Namocell for deposit of individual cells in microtiter wells.

### Example 3

### Trophoblast Enrichment From Cervical Secretions

This procedure is performed to provide a population of cells that are substantially enriched in trophoblasts, and optionally stained to distinguish between trophoblasts and maternal cells in the sample.

(A) Maternal cervical samples are collected using an endocervical brush or similar device. Paternal samples are optionally collected (saliva or 2 mL EDTA). Control samples from healthy, non-pregnant individuals are collected for spike-in experiments.

(B) Maternal gDNA, and optionally paternal gDNA, is extracted from the cell suspension on a MagNA Pure platform (Roche) using a MagNA Pure compact nucleic acid isolation kit I. If saliva from the father is used, gDNA is extracted using a MagNA Pure compact nucleic acid isolation kit I, large volume, on the same platform. Fetal cfDNA is extracted from maternal plasma on the same platform, using the large volume kit. This cfDNA is used in a Y-chromosome qPCR reaction to determine the fetal gender, based on the detection of two amplicons for DYS14 and SRY (see, e.g., A.M. Breman et al., (2016); L. Vossaert et al., (2018)).

(C) The sample is fixed by addition of 0.67× volume of 5% paraformaldehyde in PBS for 10 minutes. The sample is centrifuged (700 × g, 15 min, 4° C.) in one 5 mL tube, and the supernatant is aspirated with vacuum to waste down to a volume of 1 mL. The remaining cell pellet undergoes two washing steps with PBS, and is reduced to 1 mL in PBS. Separation buffer (PBS with 2 mM EDTA, 0.5 % BSA, 0.09% sodium azide, pH 7.2; free of Ca²⁺, Mg²⁺, and biotin) is then added.

(D) A cocktail of three biotinylated enrichment antibodies containing 4 µg of each antibody is added to each sample, and incubated for two hours at 4° C. on a laboratory shaker. The three commercial enrichment antibodies are biotinylated mouse anti-human HLA-G, biotinylated mouse anti-human TROP-2 (both Novus Biologicals), and biotinylated mouse anti-human EpCAM (BioLegend). Separation buffer (1 mL) is added to each sample, and the samples centrifuged at 400 × g. The pellet is resuspended at 10⁷ cells per 50 µL of separation buffer.

(E) Streptavidin-coated microbubbles (5 µL, Akadeum, #32211-120) are suspended by vigorous mixing until the mixture appears homogenous, then immediately added to the samples. A pipette is set to a volume equal to one half the volume of the sample-microbubble mixture, and the mixture is gently mixed by trituration using a 1000 µL low retention tip for about 30 strokes. Separation buffer (3 mL) is then added to each sample, and the samples are centrifuged (5 min, 400 × g, 4° C.). The white microbubble layer is then aspirated off, to provide a cell population enriched in fetal cells.

(F) The trophoblast cell population is optionally centrifuged, resuspended, and treated with permeabilization solution (720 µL, BD Bioscience #554723), stain reagent (250 µL, anti-cytokeratin, anti-hCG, and DAPI nuclear stain (10 µL). The resulting mixture is incubated for 1 hour at ambient temperature, flicking the tubes every 15 minutes, to provide an enriched cell population in which trophoblasts and maternal cells are labeled with contrasting stains.

(G) After immunostaining, the samples are spread into CyteSlides (RareCyte) in PBS, and subsequently scanned on a CyteScanner® (RareCyte). All identified trophoblast candidates are then curated manually, based on positive cytokeratin staining and its pattern, negative for CD45, and nuclear morphology. Using the CytePicker® module (RareCyte), all putative trophoblasts are picked individually with a 40 µm diameter needle and deposited into 200 µL PCR tubes in 2 µL PBS to provide individual, isolated fetal cells. All cells are stored at -80° C. until further processing.

### Example 4

### Nucleated Fetal Red Blood Cell Enrichment From Maternal Blood

This procedure is performed to provide a population of cells that are substantially enriched in nucleated fetal red blood cells (fnRBCs), and optionally stained to distinguish between fhRBCs and maternal cells in the sample.

(A) Maternal blood samples are collected in four 10 mL EDTA Vacutainer® tubes for trophoblast cell enrichment, and one 4 mL tube for extraction of maternal genomic DNA (gDNA) and fetal cfDNA for fetal sex determination. Paternal samples are optionally collected (saliva or 2 mL EDTA). Control samples from healthy, non-pregnant individuals are collected for lymphoblast spike-in experiments.

(B) Maternal gDNA, and optionally paternal gDNA, is extracted from whole blood on a MagNA Pure platform (Roche) using a MagNA Pure compact nucleic acid isolation kit I. If saliva from the father is used, gDNA is extracted using a MagNA Pure compact nucleic acid isolation kit I, large volume, on the same platform. Fetal cfDNA is extracted from maternal plasma on the same platform, using the large volume kit. This cfDNA is used in a Y-chromosome qPCR reaction to determine the fetal gender, based on the detection of two amplicons for DYS14 and SRY (see, e.g., A.M. Breman et al (2016); L. Vossaert et al., (2018)).

(C) The blood sample is fixed by addition of 0.67× sample volume of 5 % paraformaldehyde in PBS for 10 minutes. A cocktail of two or three biotinylated enrichment antibodies containing 4 µg of each antibody is added to each sample, and incubated for two hours at 4° C. on a laboratory shaker. The three enrichment antibodies are biotinylated mouse anti-human GPA, biotinylated mouse anti-human CD147, and biotinylated mouse anti-human transferrin receptor. Separation buffer (1 mL) is added to each sample, and the samples are centrifuged at 400 × g. The pellet is resuspended at 10⁷ cells per 50 µL of separation buffer.

(D) Streptavidin-coated microbubbles (5 µL, Akadeum, #32211-120) are suspended by vigorous mixing until the mixture appears homogenous, then immediately added to the samples. A pipette is set to a volume equal to one half the volume of the sample-microbubble mixture, and the mixture is gently mixed by trituration using a 1000 µL low retention tip for about 30 strokes. Separation buffer (3 mL) is then added to each sample, and the samples are centrifuged (5 min, 400 × g, 4° C.). The white microbubble layer is then aspirated off, to provide a cell population enriched in fetal cells.

(E) The fnRBC cell population is optionally centrifuged, resuspended, and treated with permeabilization solution (720 µL, BD Bioscience #554723), stain reagent (250 µL, anti-cytokeratin, anti-CD45) and DAPI nuclear stain (10 µL). The resulting mixture is incubated for 1 hour at ambient temperature, flicking the tubes every 15 minutes, to provide an enriched cell population in which fnRBCs and maternal cells are labeled with contrasting stains.

(F) After immunostaining, the samples are spread into CyteSlides (RareCyte) in PBS, and subsequently scanned on a CyteScanner® (RareCyte). All identified fnRBC candidates are then curated manually, based on positive cytokeratin staining and its pattern, negative for CD45, and nuclear morphology. Using the CytePicker® module (RareCyte), all putative fnRBC are picked individually with a 40 µm diameter needle and deposited into 200 µL PCR tubes in 2 µL PBS to provide individual, isolated fetal cells. All cells are stored at -80° C. until further processing.

### Example 5

### Genotyping

Before downstream analysis, each cell undergoes whole genome amplification (WGA) using the PicoPLEX WGA kit (Takara Bio). The concentration of the amplified DNA is measured using a Nanodrop platform (ThermoFisher Scientific).

All NGS is performed on WGA products from single cells, except for cell clusters. No single cells or WGA products from singleton or twin pregnancies are pooled. After consecutive DNA shearing (Covaris), end-repair (New England Biolabs reagents), A-tailing (NEB), and Illumina adaptor ligation, a round of PCR with specific Illumina primers is performed. Once the library preparation is finished, all samples are sequenced on a HiSeq2500 platform (Illumina), single-end with a read length of 100 bp, aiming for 5 × 10⁶ reads/sample. Sequence files are mapped against the human reference genome (hg19) using BWA-MEM (v.0.7.15). Coverage counts are generated with the bedtools' (v.2.25.0) multicov function.

All generated .bam files are analyzed in NxClinical (BioDiscovery). Each trophoblast is compared to a 3-cell reference pool of normal female trophoblasts and to a 3-cell reference pool of normal male trophoblasts for genome-wide CNV analysis. This software enables multiple modes of data visualization, including whole genome plots for each cell that allow for nucleotide-level zooming in on the data and multi-sample views indicating the copy number changes automatically called within the software. The results are compared to consensus / normal sequences to identify any variations that are associated with pathology.

A multiplex PCR including 41 amplicons is run on the WGA products and maternal gDNA (as well as paternal when available), and the resulting products sequenced by MiSeq sequencing (2 × 150 bp reads, paired-end). The SNP profiles are subsequently compared with the maternal profile. In case a fetal allele for a given SNP is different from the maternal profile, this confirms the fetal origin of the cell.

An alternative genotyping method is to score SNP alleles from individual cells and compare these back to complete SNP genotyping on maternal genomic DNA; the number of SNP alleles in the candidate fetal cell that are not present in the mother is then determined. If 5-10 million NGS reads are analyzed, there are typically 2,000-6,000 alleles detected in each single fetal cell that are not present in the mother. If the cell is maternal, there should be no alleles in the cell that are not present in the mother’s genomic DNA, other than genotyping artifacts and other noise.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A method for preparing a cell population enriched in fetal cells, the method comprising:
(a) providing a biological sample from a pregnant subject;
(b) contacting the sample with
(i) at least one binding agent, wherein the binding agent comprises a targeting moiety specific for a fetal cell antigen; and
(ii) a buoyant microbubble, wherein the binding agent binds to the microbubble surface; to form fetal cell-binding agent-microbubble complexes, wherein the complexes have an average ratio of fetal cell to microbubble of about 1:5 to about 5:1, and have an average density of from about 40% to about 80% of the average density of the sample;
(c) separating the fetal cell-binding agent-microbubble complexes from other cells in the sample; and
(d) collecting the fetal cell-binding agent-microbubble complexes from the sample, to provide a cell population enriched in fetal cells.

2. The method of claim 1, wherein step (d) further comprises releasing fetal cells from the complexes.

3. The method of claim 1 or 2, wherein the fetal cell is a trophoblast.

4. The method of claim 1 or 2, wherein the fetal cell is a nucleated fetal red blood cell.

5. The method of any one of claims 1 to 4, wherein the binding agent further comprises a first linking moiety, and the microbubble further comprises a second linking moiety, where the first linking moiety and the second linking moiety specifically bind to each other.

6. The method of any one of claims 1 to 5, wherein the first linking moiety and the second linking moiety have a dissociation constant (K_{D}) for each other of from about 10⁻⁶ to about 10⁻¹⁶.

7. The method of claim 6, wherein the K_{D} is from about 10⁻⁸ to about 10⁻¹⁵.

8. The method of any one of claims 5 to 7, wherein the first linking moiety and the second linking moiety are each selected from the group consisting of antibodies, antibody derivatives, antigens, biotin, avidin, and streptavidin.

9. The method of any one of claims 1 to 8, wherein the first linking moiety and the second linking moiety are each selected from the group consisting of biotin, avidin, and streptavidin.

10. The method of any one of claims 1 to 8, wherein the second linking moiety comprises an antibody or antibody derivative, and the first linking moiety is an epitope on the targeting moiety.

11. The method of any one of claims 1 to 9, wherein the microbubble has a diameter between about 10 µm and about 20 µm.

12. The method of any one of claims 1 to 11, wherein the microbubble has a diameter between about 13 µm and about 19 µm.

13. The method of any one of claims 1 to 12, wherein the microbubble has a diameter between about 16 µm and about 18 µm.

14. The method of any one of claims 1 to 13, wherein the microbubble has a density of about 0.4 g/cm³ and about 0.8 g/cm³.

15. The method of any one of claims 1 to 14, wherein the microbubble has a density of about 0.6 g/cm³.

16. The method of any one of claims 1 to 15, wherein the microbubble is hollow.

17. The method of any one of claims 1 to 16, wherein the microbubble comprises glass.

18. The method of any one of claims 1 to 17, wherein the fetal cell-binding agent-microbubble complex rises in the sample at a rate of about 1 mm/min to about 15 mm/min at 1 × g.

19. The method of any one of claims 1 to 18, wherein the fetal cell-binding agent-microbubble complex rises in the sample at a rate of about 5 mm/min to about 10 mm/min at 1 × g.

20. The method of any one of claims 1 to 19, wherein step (c) comprises centrifuging the sample.

21. The method of claim 20, wherein the sample is centrifuged at about 200 × g to about 800 × g.

22. The method of claim 21, wherein the sample is centrifuged at about 400 × g to about 500 × g.

23. The method of any one of claims 20 to 22, wherein the sample is centrifuged for about 2 minutes to about 10 minutes.

24. The method of claim 23, wherein the sample is centrifuged for about 5 minutes.

25. The method of any one of claims 1 to 24, wherein the biological sample comprises a blood sample.

26. The method of any one of claims 1 to 24, wherein the biological sample comprises a cervical secretion.

27. The method of any one of claims 1 to 24, wherein the biological sample comprises amniotic fluid.

28. The method of claim 25, wherein step (c) comprises removing erythrocytes from the sample by lysis.

29. The method of claim 28, wherein the erythrocytes are lysed using ammonium chloride or a surfactant.

30. The method of claim 28 or 29, wherein the erythrocytes are lysed using Triton X-100®.

31. The method of any one of claims 1 to 30, wherein the targeting moiety comprises an antibody.

32. The method of claim 31, wherein the antibody is specific for an antigen selected from the group consisting of HLA-G, EpCAM, and TROP-2.

33. The method of any one of claims 1 to 32, wherein step (c) comprises contacting the sample with at least two binding agents having different targets.

34. The method of any one of claims 1 to 33, wherein step (c) comprises contacting the sample with a plurality of binding agents having different targets.

35. The method of claim 34, wherein binding agents specific for each of HLA-G, EpCAM, and TROP-2 are used.

36. The method of any one of claims 5 to 35, wherein the microbubble is first contacted with a binding agent to form a binding agent-microbubble complex, and the fetal cell is then contacted with the binding agent-microbubble complex to form a fetal cell-binding agent-microbubble complex.

37. The method of any one of claims 5 to 35, wherein the fetal cell is first contacted with a binding agent to form a fetal cell-binding agent complex, and then the fetal cell-binding agent complex is contacted with a microbubble to form a fetal cell-binding agent-microbubble complex.

38. The method of any one of claims 1 to 37, wherein the sample is contacted with a label.

39. The method of claim 38, wherein the label is selected from the group consisting of: 4',6-diamidino-2-phenylindole (DAPI) or a labeled binding agent specific for an antigen selected from the group consisting of HLA-E, HLA-G, MCAM, ATG9B, EpCAM, TROP-2, CD144, CD47, transferrin receptor (CD71), thrombospondin receptor (CD36), glycophorin A, CD147, and CD45.

40. The method of claim 38 or 39, wherein the label is DAPI or a labeled binding agent specific for an antigen selected from the group consisting of cytokeratin and CD45.

41. The method of any one of claims 38 to 40, wherein the sample is contacted with a permeabilization agent prior to contact with the label.

42. The method of any one of claims 38 to 41, wherein the sample is contacted with a label after step (c).

43. The method of any one of claims 1 to 42, wherein step (d) comprises aspirating the fetal cell-binding agent-microbubble complex from the sample surface.

44. The method of any one of claims 1 to 43, wherein step (d) further comprises diluting the fetal cell-binding agent-microbubble complex to provide aliquots having 0 or 1 cells.

45. The method of claim 44, wherein the aliquots are contained in separate wells.

46. The method of claim 44, wherein the aliquots are contained in separate droplets.

47. The method of any one of claims 1 to 46, wherein step (d) further comprises isolating the fetal cell using a single-cell picking device.

48. The method of any one of claims 1 to 47, wherein the cell population enriched in fetal cells has a ratio of fetal cell to other cells of at least about 1:100, at least about 1:20, at least about 1:10, at least about 1:5, about 1:1, or greater than about 1:1.

49. The method of any one of claims 1 to 48, wherein the biological sample is obtained from the subject at a gestational age of less than about 17, 16, 15, 14, 13, 12, 11, 10, 9, 8, 7, 6, 5, or 4 weeks.

50. The method of claim 49, wherein the gestational age is between about 6 weeks and about 15 weeks.

51. The method of any one of claims 1 to 50, wherein the sample is fixed with formaldehyde or glutaraldehyde.

52. The method of any one of claims 1 to 51, wherein at least about 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 80, or 100 fetal cells are obtained per sample in condition for NGS.

53. The method of any one of claims 1 to 52, wherein at least about 5, 10, 20, 25, 30, 35, 40, 45, or 50 fetal cells are obtained from at least about 70, 75, 80, 85, 90, 95, 96, 97, 98, or 99% of samples from the subjects tested.

54. A method for obtaining nucleic acids from fetal cells present in a biological sample obtained during pregnancy, the method comprising:
(a) providing a cell population enriched in fetal cells by the method of any one of claims 1 to 51; and
(b) lysing the fetal cells to obtain the nucleic acids.

55. The method of claim 54, wherein the fetal cells are lysed as a pool of about 5 fetal cells or fewer.

56. The method of claim 54 or 55, wherein the fetal cells are lysed as a pool of less than about 4, 3, or 2 fetal cells.

57. The method of any one of claims 54 to 56, wherein the fetal cells are lysed individually.

58. The method of any one of claims 54 to 57, wherein the cell population enriched in fetal cells comprises fetal cells at a ratio of fetal cells to maternal cells of about 1:5 to about 5:1.

59. The method of claim 58, wherein the ratio is about 1:1.

60. The method of any one of claims 54 to 59, wherein both fetal and maternal cells are lysed individually.

61. A method for genotyping a fetus, the method comprising:
(a) providing fetal cell nucleic acids by the method of any one of claims 54 to 60;
(b) amplifying the nucleic acids; and
(c) detecting an indication of a genetic difference.

62. The method of claim 61, wherein the indication is a copy number variation of a gene or a chromosomal region.

63. The method of claim 62, wherein the chromosomal region is less than about 2 Mb in length.

64. The method of claim 62 or 63, wherein the chromosomal region is less than about 1, about 0.9, about 0.8, about 0.7, about 0.6, about 0.5, about 0.4, about 0.3, about 0.2, or about 0.1 Mb in length.

65. The method of claim 61, wherein the indication is a copy number variation of substantially an entire chromosome.

66. The method of claim 61, wherein the indication is a translocation.

67. The method of claim 61, wherein the indication is a nucleic acid sequence associated with a pathological condition.

68. The method of claim 67, wherein the nucleic acid sequence associated with a pathological condition is an allele associated with a pathological condition.

69. The method of claim 61, wherein the indication is a polymorphism.

70. The method of claim 69, wherein the polymorphism is an indel.

71. The method of claim 61 or 69, wherein the indication is a single nucleotide polymorphism (SNP).

72. The method of claim 61, wherein the genetic difference is mosaicism.

73. The method of claim 72, wherein the mosaicism is confined placental mosaicism.

74. The method of claim 61, wherein the genetic difference is uniparental disomy.

75. The method of claim 61, wherein the genetic difference is twins.

76. The method of claim 61, wherein the pathological condition is 1p36 deletion syndrome, 18p deletion syndrome, 21-hydroxylase deficiency, Alpha 1-antitrypsin deficiency, AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, Alagille syndrome, ADULT syndrome, Aicardi-Goutieres syndrome, Albinism, Alexander disease, alkaptonuria, Alport syndrome, Alternating hemiplegia of childhood, Amyotrophic lateral sclerosis - Frontotemporal dementia, Alström syndrome, Amelogenesis imperfecta, Aminolevulinic acid dehydratase deficiency porphyria, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Björnstad syndrome, Bloom syndrome, Birt-Hogg-Dube syndrome, Brody myopathy, Brunner syndrome, CADASIL syndrome, CARASIL syndrome, Chronic granulomatous disorder, Campomelic dysplasia, Canavan disease, Carpenter Syndrome, Cerebral dysgenesis-neuropathy-ichthyosis-keratoderma syndrome (SEDNIK), Cystic fibrosis, Charcot-Marie-Tooth disease, CHARGE syndrome, Chediak-Higashi syndrome, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn’s disease, Crouzon syndrome, Crouzonodermoskeletal syndrome (Crouzon syndrome with acanthosis nigricans), Darier’s disease, Dent’s disease (Genetic hypercalciuria), Denys-Drash syndrome, De Grouchy syndrome, Down Syndrome, Di George’s syndrome, Distal hereditary motor neuropathies, multiple types, Distal muscular dystrophy, Duchenne muscular dystrophy, Dravet syndrome, Edwards Syndrome, Ehlers-Danlos syndrome, Emery-Dreifuss syndrome, Epidermolysis bullosa, Erythropoietic protoporphyria, Fanconi anemia (FA), Fabry disease, Factor V Leiden thrombophilia, Fatal familial insomnia, Familial adenomatous polyposis, Familial dysautonomia, Familial Creutzfeld-Jakob Disease, Feingold syndrome, FG syndrome, Fragile X syndrome, Friedreich’s ataxia, G6PD deficiency, Galactosemia, Gaucher disease, Gerstmann-Sträussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary spastic paraplegia (infantile-onset ascending hereditary spastic paralysis), Hermansky-Pudlak syndrome, Hereditary neuropathy with liability to pressure palsies (HNPP), Heterotaxy, Homocystinuria, Huntington’s disease, Hunter syndrome, Hurler syndrome, Hutchinson-Gilford progeria syndrome, Hyperlysinemia, Hyperoxaluria, primary, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Kniest dysplasia, Kosaki overgrowth syndrome, Krabbe disease, Kufor-Rakeb syndrome, LCAT deficiency, Lesch-Nyhan syndrome, Li-Fraumeni syndrome, Limb-Girdle Muscular Dystrophy, Lynch syndrome, lipoprotein lipase deficiency, Malignant hyperthermia, Maple syrup urine disease, Marfan syndrome, Maroteaux-Lamy syndrome, McCune-Albright syndrome, McLeod syndrome, MEDNIK syndrome, Mediterranean fever, familial, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer’s syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Porphyria cutanea tarda (PCT), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Sjogren-Larsson syndrome, Spondyloepiphyseal dysplasia congenita (SED), Shprintzen-Goldberg syndrome, Sickle cell anemia, Siderius X-linked mental retardation syndrome, Sideroblastic anemia, Sly syndrome, Smith-Lemli-Opitz syndrome, Smith-Magenis syndrome, Snyder-Robinson syndrome, Spinal muscular atrophy, Spinocerebellar ataxia (types 1-29), SSB syndrome (SADDAN), Stargardt disease (macular degeneration), Stickler syndrome (multiple forms), Strudwick syndrome (spondyloepimeta-physeal dysplasia, Strudwick type), Tay-Sachs disease, Tetrahydrobiopterin deficiency, Thanatophoric dysplasia, Treacher Collins syndrome, Trisomy 8, Trisomy 9, Trisomy, 22, Tuberous sclerosis complex (TSC), Turner syndrome, Usher syndrome, Variegate porphyria, von Hippel-Lindau disease, Waardenburg syndrome, Weissenbacher-Zweymüller syndrome, Williams syndrome, Wilson disease, Woodhouse-Sakati syndrome, Wolf-Hirschhorn syndrome, Xeroderma pigmentosum, X-linked intellectual disability and macroorchidism (fragile X syndrome), X-linked spinal-bulbar muscle atrophy (spinal and bulbar muscular atrophy), Xp11.2 duplication syndrome, X-linked severe combined immunodeficiency (X-SCID), X-linked sideroblastic anemia (XLSA), 47,XXX (triple X syndrome), XXXX syndrome (48, XXXX), XXXXX syndrome (49, XXXXX), XYY syndrome (47,XYY), or Zellweger syndrome.

77. The method of claim 76, wherein the pathological condition is selected from the group consisting of: Angelman syndrome, Canavan disease, Charcot-Marie-Tooth disease, Cri du chat syndrome, Cystic fibrosis, DiGeorge syndrome, Down syndrome, Duchenne muscular dystrophy, Familial hypercholesterolemia, Haemochromatosis, Hemophilia, Klinefelter syndrome, Neurofibromatosis, Phenylketonuria, Polycystic kidney disease (PKD1 or PKD2, Prader-Willi syndrome, Sickle cell disease, Spinal muscular atrophy, Tay-Sachs disease, and Turner syndrome.

78. The method of any one of claims 61 to 77, wherein the nucleic acids from fetal cells are amplified as a pool of about 5 fetal cells or less.

79. The method of claim 78, wherein the pool of fetal cells has 4, 3, 2, or 1 fetal cell.

80. The method of claim 79, wherein the pool of fetal cells has about 1 fetal cell.

81. The method of any one of claims 61 to 80, wherein step (b) comprises whole genome amplification.

82. The method of any one of claims 61 to 81, wherein step (c) comprises quantitative polymerase chain reaction amplification (qPCR), array comparative genomic hybridization (array CGH), or next generation sequencing (NGS).

83. The method of claim 82, wherein NGS is single cell NGS.

84. The method of claim 82 or 83, wherein NGS is used at a depth of at least 20 X.

85. The method of any one of claims 82 to 84, wherein NGS is used at a depth of at least 22X, 23X, 24X, 25X, 26X, 27X, 28X, 29X, or 30 X.

86. A system for obtaining nucleic acids from fetal cells present in a maternal biological sample during pregnancy, the system comprising:
(a) at least one binding agent, wherein the binding agent comprises a targeting moiety specific for a fetal cell antigen; and
(b) a buoyant microbubble, wherein the binding agent binds to the microbubble surface; wherein the binding agent and microbubble form fetal cell-binding agent-microbubble complexes, wherein the complexes have an average ratio of fetal cell to microbubble of about 1:5 to about 5:1, and wherein the microbubble has a density of about 0.4 g/cm³ and about 0.8 g/cm³.

87. The system of claim 86, further comprising a label.

88. The system of claim 87, wherein the label is specific for fetal cells.

89. The system of any one of claims 86 to 88, wherein the microbubble has a diameter between about 10 µm and about 20 µm.

90. The system of claim 89, wherein the microbubble has a diameter between about 13 µm and about 19 µm.

91. The system of claim 90, wherein the microbubble has a diameter between about 16 µm and about 18 µm.

92. The system of claim 91, wherein the microbubble has a density of about 0.6 g/cm³.

93. A fetal cell composition, comprising a fetal cell-binding agent-microbubble complex as described in any one of claims 1 to 51 and maternal cells, wherein the ratio of fetal cells to maternal cells is from about 1:1,000 to about 1,000:1.

94. The composition of claim 93, wherein the ratio is from about 1:100 to about 5:1.

95. The composition of claim 93 or 94, wherein the ratio is from about 1:10 to about 1:1.

96. The composition of any one of claims 93 to 95, wherein the fetal cell is a circulating trophoblast or a fetal nucleated red blood cell.

97. The composition of any one of claims 93 to 96, wherein the ratio of fetal cell to microbubble is about 5:1 to about 1:5.

98. The composition of any one of claims 93 to 97, wherein the ratio of fetal cell to microbubble is about 1:1.

99. A fetal cell-enriched cell population, comprising fetal cells as described in any one of claims 2 to 50 and maternal cells, wherein the ratio of fetal cells to maternal cells is from about 1:1,000 to about 1,000:1.

100. The composition of claim 99, wherein the ratio is from about 1:100 to about 5:1.

101. The composition of claim 99 or 100, wherein the ratio is from about 1:10 to about 10:1.

102. The composition of any one of claims 99 to 101, wherein the fetal cell is a circulating trophoblast or a fetal nucleated red blood cell.

103. The composition of any one of claims 93 to 102, wherein the fetal cells are stained.

104. The composition of any one of claims 93 to 103, wherein the composition is obtained by the method of any one of claims 1 to 51.
